(19) United States
(12) Patent Application Publication
(10) Pub. No.: US 2025/0022362 A1
(43) Pub. Date: Jan. 16, 2025

(54) A WIRELESS, HIGH-RESOLUTION, AND SMARTWATCH-COMPATIBLE WEARABLE DATA READOUT SYSTEM FOR LOW-VOLTAGE TRANSISTOR CHARACTERIZATION

(71) Applicant: The University of Hong Kong, Hong Kong (CN)

(72) Inventors: Shiming ZHANG, Hong Kong (CN); Xinyu TIAN, Hong Kong (CN); Kwok Leung CHAN, Hong Kong (CN); Dingyao LIU, Hong Kong (CN); Jing BAI, Hong Kong (CN)

(73) Assignee: The University of Hong Kong, Hong Kong (CN)

(21) Appl. No.: 18/714,474

(22) PCT Filed: Nov. 30, 2022
(86) PCT No.: PCT/CN2022/135324

§ 371 Date: May 29, 2024

(51) Int. Cl. G08C 17/02; G04G 21/04; A61B 5/00; H10K 10/46

(52) U.S. Cl. CPC G08C 17/02; G04G 21/04; A61B 5/681; A61B 5/685; H10K 10/46

(57) ABSTRACT

An electronic reader for electro-chemical transistors (OECT) includes a potential output control module that controls the Vd, Vs, and Vg for the OECT under test, a high accuracy current monitor module which contains a transimpedance amplifier (TIA) used to control the output voltage and convert the input channel current Ids into a voltage value. A microcontroller (MCU) that controls the working sequences of the TIA so as to realize the specific characterization mode and enable an adjustable output voltage range. The MCU further controls a programmable sampling rate of up to 200 k samples per second (SPS) with low noise by down-sampling the base rate, convolution processing it and interpolating it back to ah high frequency, but without the noise. The device is small enough to be worn by a user and its output is sent to a mobile device for reading.

### CROSS-REFERENCE TO RELATED PATENT APPLICATIONS

This application is a U.S. National Stage Application under 35 U.S.C. § 371 of International Patent Application No. PCT/CN2022/135324, filed Nov. 30, 2022, and claims the benefit of priority under 35 U.S.C. Section 119(e) of U.S. Application No. 63/284,211, filed Nov. 30, 2021, all of which are incorporated herein by reference in their entireties. The International Application was published on Jun. 8, 2023 as International Publication No. WO 2023/098709 A1.

### FIELD OF THE INVENTION

The present invention relates to systems for characterizing low voltage transistors and, more particularly, to a wireless, high-resolution, and smartwatch-compatible wearable data readout system for low-voltage transistor characterization.

### BACKGROUND OF THE INVENTION

Low-voltage transistors, especially organic electrochemical transistors (OECT), are regarded as the next-generation of biosensing technology because of their ultrahigh sensitivity, water stability and cost-effectiveness.

The current OECT data characterization system is poorly integrated and is large in size (>20 cm*10 cm*5 cm). Further, resolution and sampling rate are low (>1 uA), resulting in an insufficient sampling ability for qualified biosensing.

One article disclosing a portable OECT sensing system is Ji, X; Lau, H. Y.; Ren. X.; Peng B.; Zhai, P.; Feng, S.-P.; Chan, P. K. L., Advanced Materials Technologies 2016, 1, 1600042. The device size is described in the article as around 60 mm length*40 mm width*25 mm thickness. It cannot be integrated with a smartwatch. Another article discloses an electrochemical detection system in which the size of the detector is 80 mm length*40 mm width*23 mm thickness. Ainla, A.; Mousavi, M. P. S.; Tsaloglou, M.-N.; Redston, J.; Bell, J. G.; Fernindez-Abedul, M. T.; Whitesides, G. M., Analytical Chemistry 2018, 90, 6240-6246. It cannot be integrated with a smartwatch and cannot measure an OECT. The present inventors previous work demonstrated an OECT sensing system compatible with a watch. Zhang, S.; Ling, H.; Chen, Y.; Cui, Q.; Ni, J.; Wang, X.; Hartel, M. C.; Meng, X.; Lee, K.; Lee, J.; Sun, W.; Lin, H.; Emaminejad, S.; Ahadian, S.; Ashammakhi, N.; Dokmeci, M. R.; Khademhosseini, A., Advanced Functional Materials 2020, 30, 1906016. However, the resolution of the previous work was low (>10 uA) and the sampling rate was low (2K). Besides, it was of a low integration level and cannot be used to characterize the overall performance of an OECT.

A further article disclosed an OECT sensing system whose size is around 60 mm in length*40 mm width*25 mm thickness. Liu, H.; Yang, A.; Song, J.; Wang, N.; Lam, P.; Li, Y.; Law, H. K.-W.; Yan, F., Science Advances 2021, 7. However, it cannot be integrated with a smartwatch. Each of the articles in the prior two paragraphs is incorporated herein by reference in its entirety.

Thus, a wireless, smartwatch-compatible, and high-resolution readout unit that can characterize the overall performance of an OECT is still unavailable, which hinders the assembly of truly integrated OECT systems for wearable bio-sensing applications. Even more so, a miniaturized OECT characterization system that can be integrated with a smartwatch is not available, which hinders the development of sensors with OECTs that can be worn on a user's wrist. In addition, an OECT-based fully-integrated wearable platform is still missing, which hinders the development of biosensing applications, such as microneedle-based sensors and brain-probe-based sensors. Besides, the absence of such a system also hinders the development of wearable computing devices with OECT.

### SUMMARY OF THE INVENTION

The present invention is a new product: the world's smallest (coin-sized) readout unit for remote and wireless OECT characterization. It is a “personalized electronic reader for electrochemical transistors” (PERfECT). The novel PERfECT platform can be embedded into a smartwatch and can measure the overall performance of OECT devices. Besides, it is also capable of measuring a number of other kinds of electrochemical transducers. The resolution of data acquisition is on the level of a nano-ampere, which is comparable with laboratory-based commercial bulky equipment. The PERfECT system is adjustable for extended uses in digital healthcare, wearable health, brain-inspired neuromorphic computing and edge computing applications.

The PERfECT system can measure the transfer, output, hysteresis and transient behaviors of OECT, with resolution and sampling rates that can be benchmarked to the bulky equipment used in laboratories. The present invention paves the way for the development of OECT-based medical devices for truly wearable healthcare monitoring applications.

The OECT characterization system (PERfECT), whose dimensions are as small as a smartwatch (<1.5 cm*1.5 cm*0.5 cm), enables its use for the integration of truly wearable applications. In order to accomplish this state-of-the-art microelectronic technologies and advanced circuit design strategies are used to endow PERfECT with a high resolution. The combination of small size (smartwatch scale), high resolution (nA) and high sampling speed (>200 k SPS) is novel. The system can serve as a fundamental building block for the prototyping of various smart wearable sensing systems, such as micro-needle biosensing and brain probes. Besides, it can serve as a platform on which wearable edge computing can be developed.

In an exemplary embodiment the system of the present invention employs four sub-modules, i.e., i) a potential output control module that contains three digital-to-analog converters (DAC) and potentiostat amplifiers (PA) to control the Vd, Vs, and V_{g}, ii) a high accuracy current monitor module which contains a trans-impedance amplifier (TIA) and an analog-to-digital converter (ADC) to control the output voltage and convert the input channel current Ids into a voltage value, iii) a microcontroller (MCU) that controls the working sequences of the DAC, TIA, and ADC to realize the specific characterization mode and enable an adjustable output voltage range, and iv) is a wireless communication module which is used to connect with a mobile device for data exchange and transmission.

The MCU executes a programmable sampling rate of up to 200K samples per second (SPS) and may also execute a digital noise filter program. Also, by taking advantage of the ultra-low power consumption, a Bluetooth-Low-Power (BLE) chip DA14585 can be used for communications. After integrating all these modules, the invention has a dimension less than 1.5 cm*1.5 cm, which benefits its uses for wearable applications.

Moreover, PERfECT is equipped with major functions of an electrochemical (EC) workstation such as cyclic voltammetry (CV), amperometry, potentiometry, and electrochemical impedance spectroscopy (EIS). These merits, altogether, make PERfECT a highly desirable unit that allows the assembling of remote health motoring devices based on EC and OECT technologies.

### BRIEF DESCRIPTION OF THE DRAWINGS

The foregoing and other objects and advantages of the present invention will become more apparent when considered in connection with the following detailed description and appended drawings in which like designations denote like elements in the various views, and wherein:

FIG. 1A illustrates the typical structure of an OECT and a schematic diagram of a device characterizing the structure of an OECT and FIG. 1B provides graphs of the characterizations of an OECT, i.e., the transfer curve, output curve, hysteresis loop, and transient responses;

FIG. 2A shows optical photos of a design of a PERfECT system according to the present invention and a smartphone running an application for reading the system output, FIG. 2B shows the circuit diagram and main components of PERfECT and FIG. 2C is a photograph showing how PERfECT works in wearable applications;

FIG. 3A is a graphs characterizing the transfer curves of the OECTs, FIG. 3B shows the related transconductance, FIG. 3C shows the output characterizations and FIG. 3D shows the hysteresis characterization of the PERfECT system of the present invention;

FIG. 4A illustrates the transient characterization of the output of the present invention showing the current responses (lower curves) under different widths and amplitude gate pulses (upper curves) and FIG. 4B shows the OECT current responses under a group of 1 ms pulses;

FIG. 5A shows a a graph of the current charge for a PERfECT-based prototype for wearable sensing with respect to time and FIG. 5B shows the current with respect to concentration;

FIG. 6A is a comparison between the present invention and the prior art showing the type of systems and FIG. 6B showing the parameters for various prior systems and the present invention;

FIG. 7 shows an arrangement and equations for using a negative feedback network to stabilize applied voltage;

FIG. 8 is a block diagram of a complementary filtering method of the present invention for increasing the sampling rate;

FIG. 9A is a schematic diagram of a system for electronic validation of the invention and FIG. 9B is a graph of current output in pA; and

FIG. 10 is an electrochemistry test comparison between a prior art device and the present invention.

### DETAILED DESCRIPTION OF THE INVENTION

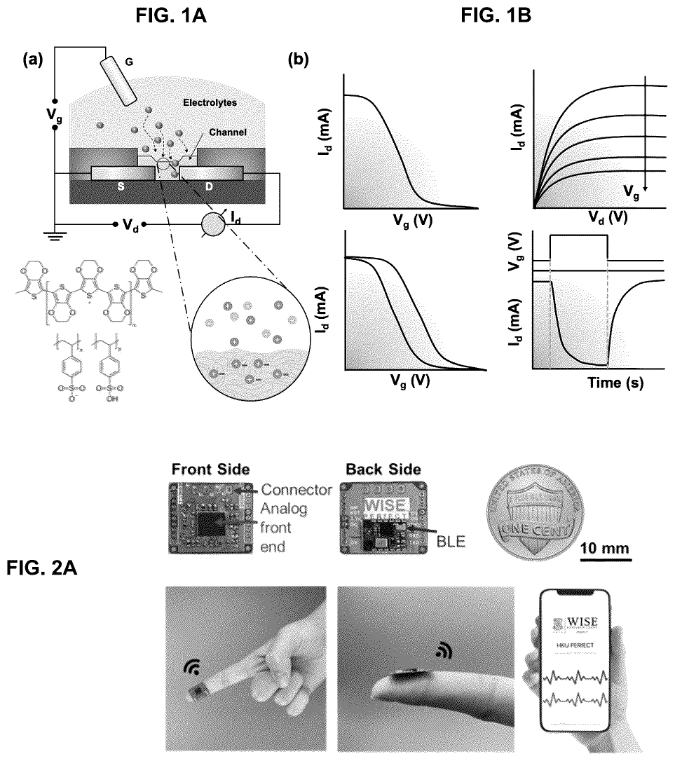

Portable and wearable biosensors are in high demand in order to achieve the goal of decentralized and personalized healthcare. [1] Organic electrochemical transistors (OECTs), which combine the advantages of both an electrochemical cell and a microelectronic transistor, have high signal amplification ability and are energy efficient. [2-4] The conventional OECT device structure is illustrated in FIG. 1A. It consists of source (S)-drain (D) electrodes, an organic semiconductor channel, an electrolyte, and a gate electrode. The organic semiconductor channel, such as poly(3,4-ethylenedioxythiophene) polystyrene sulfonate (PEDOT:PSS), is in direct contact with the electrolyte in which the gate electrode is immersed. When a positive (negative) gate voltage (V_{gs}) is applied, the cations (anions) in the electrolyte are electrostatically repulsed into the channel, and an electrochemical doping (dedoping) process subsequently occurs. This doping (dedoping) process changes the conductivity of the channel. In this way, a slight change in Vgs is converted to a more considerable change in source-drain current (I_{ds}) due to the amplification ability of OECTs, which so far has the highest transconductance (Gm) among all transistors. [5] Therefore, OECTs have been widely investigated for use in applications in emerging research areas, including health monitoring [6-11], bioelectronic interfacing [12-14], and neuromorphic computing [15-17]. However, despite these advantages, a miniaturized OECT characterization system is still not available. Such a device is an indispensable building block in the development of fully integrated OECTs for portable and wearable applications. The development of a miniaturized OECT characterization system is challenging because it requires transdisciplinary efforts among microelectronics, embedded systems, wireless communication, software engineering, and device engineering.

The present invention has resulted in the world's smallest miniaturized system to date, which is referred to as a “personalized electronic reader for electro-chemical transistors” (PERfECT), for wearable and portable OECT characterization. By selecting a best-fit commercial chip, the system is enabled for electrochemical characterization. Also, synergistic and collaborative efforts between circuit design, software engineering, and device engineering have been utilized to arrive at the invention.

The present invention incorporates a universal method to increase the readout resolution by introducing a negative feedback network. In addition, an efficient software algorithm is used to increase the sampling rate. The resultant PERfECT system is a coin-sized module, which can measure an OECT wirelessly and is controllable with a customized application (APP) on a mobile device. In addition to OECTs, experimental validation demonstrates that PERfECT is also capable of characterizing other kinds of electrolyte-gated transistors. The preeminence of PERfECT is comparable to that of bulky equipment used in a laboratory. Moreover, PERfECT is equipped with major functions of an electrochemical (EC) workstation such as cyclic voltammetry (CV), amperometry, potentiometry, and electrochemical impedance spectroscopy (EIS). These merits, altogether, make PERfECT a highly desirable unit that allows the assembling of remote health motoring devices based on EC and OECT technologies.

In carrying out the present invention OECTs are fabricated on plastic substrates (3M Tegaderm Roll). The source, drain, and gate electrodes are pre-patterned on plastic substrates in a planar structure. Then, a PEDOT:PSS suspension is spin-coated and patterned between the source and drain electrodes. Kapton tape is used as a show mask for the patterning of the channel. Prior to spin-coating, the PEDOT:PSS suspension is mixed with surfactant dodecyl benzene sulfonic acid (DBSA) (0.5 v/v. %) and crosslinker 3-glycidoxypropyltrimethoxysilane (GOPS) (1 v/v. %) to improve the wettability and adhesion on the substrate. It is then baked on a hotplate for 1 hour to anneal the PEDOT:PSS channel. Afterward, the devices are soaked in deionized water to remove saline contaminants from the PEDOT:PSS film, while a solid-state ion gel is subsequently used as an electrolyte bridging the gate electrode and the PEDOT:PSS channel.

The ion gel is prepared through a one-step polymerization, in which a zwitterionic monomer 3-dimethyl (methacryloyloxyethyl) ammonium propane sulfonate (DMAPS) is first mixed with ionic liquid (1-ethyl-3-methylimidazolium ethyl sulfate) and deionized water with a weight ratio of 1:1:4.67, and then initialized by ammonium persulfate (APS) at 70° C. for 6 hours. Afterwards it is cured in a 50° C. oven to remove excess water and to obtain an ion gel.

FIG. 1B shows transfer, output, hysteresis loop and transient response of the device. The OECTs characterizations were performed with both the PERfECT system and commercial equipment, Agilent Keithley B2902A.

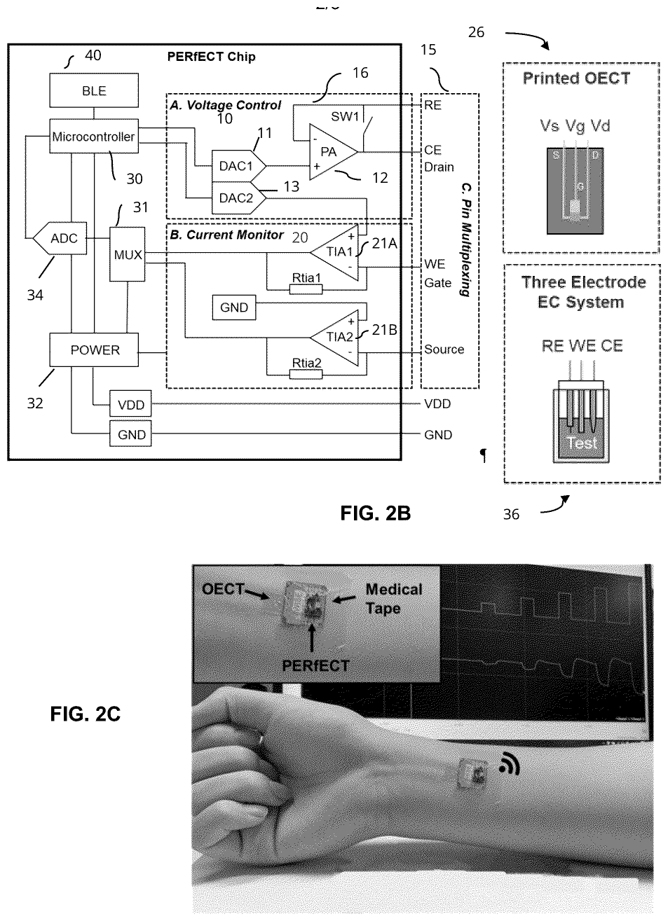

FIG. 2A shows an integrated circuit design of PERfECT on a printed circuit board (PCB). The layout was designed with free, open-source electronic design automation software, KiCad 5.1.10 (www.kicad.org). After the circuit lay-out was validated with design rule check, a commercial PCB manufacturer (JDBPCB) fabricated the flexible printed circuit board (fPCB) on a thin, flexible film of multilayer copper/PI structure. All the components were ordered from DigiKey (MN, USA) and hand-soldered to the fPCB board via soldering iron tip and heat gun. The circuit was programmed by the Joint Test Action Group (JTAG) interface, and the firmware of the circuit was developed in C language. Multi-layer manufacturing processes were used to decrease the dimension of the fPCB board to ease its use for wearable applications.

The previously demonstrated system for OECT characterization is of low resolution and low sampling speed. Besides, it cannot characterize the dynamic performance of the OECT. The following challenges cause this bottleneck issue:
1. The lack of a methodology to precisely control the voltage of the OECTs because of the voltage shift caused by redox reactions with the electrolyte, including the lack of a miniaturized integrated circuit to precisely control the voltage of OECTs during the channel doping and dedoping processes.
2. The lack of a methodology to prevent current leakage of the OECT, which results in a low readout resolution, including the lack of a miniaturized integrated circuit to readout the small current from the OECT with high resolution.
3. The lack of an algorithm to increase the sampling rate to meet the specific requirement of the OECTs for practical applications.
4. The lack of a methodology to operate electrochemical (EC)-OECT dual mode measurement with one single chip.

To solve these challenges, the present invention uses a fully integrated chip (FIG. 2B) which can achieve full function characterization of low-voltage transistors, especially for organic electrochemical transistors. Three key modules are designed and integrated into the PERfECT chip to address the challenges.

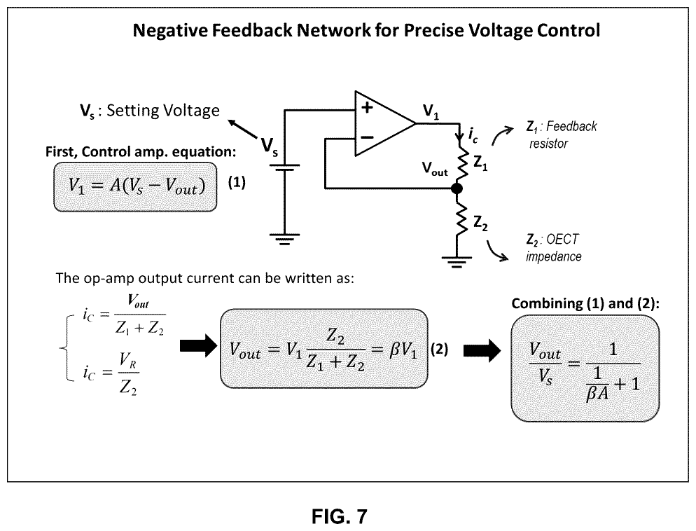

In solving challenge 1, the integrated circuit includes a transistor voltage control module 10 that is integrated into the PERfECT chip. The transistor voltage control module is designed by following two methods: (1) the use of a negative feedback network (NFN) to precisely control the voltage of a reference electrode RE and (2) introducing a transimpedance amplifier (TIA) 21A, 21B as a current monitor 20 to avoid current leakage, where the TIA provide a dynamic impedance to match the input impedance. Conventional approaches use a voltage supply to directly control the reference voltage, which causes instability issues because of the unexpected voltage shift in an aqueous environment. In this new method, a reference electrode is introduced into the circuits. By employing the specifically designed negative feedback network (NFN, detailed in FIG. 7), the voltage of the reference electrode can be precisely controlled. The NFN allows a dynamic verification of the gate voltage and ensures a high resolution.

To solve challenge 2, the present invention uses the following method: Conventional approaches use a load resistor to control the source-drain voltage, which fails to provide high resolution due to the leakage current generated by the inherent impedance mismatch in the circuits. To solve this challenge, the present invention uses a novel approach by designing and introducing the transimpedance amplifier (TIA) 21A, 21B to avoid current leakage (FIG. 2B). As noted, the TIA provides a dynamic impedance to match the input impedance, which helps minimize the leakage current, which improves the resolution.

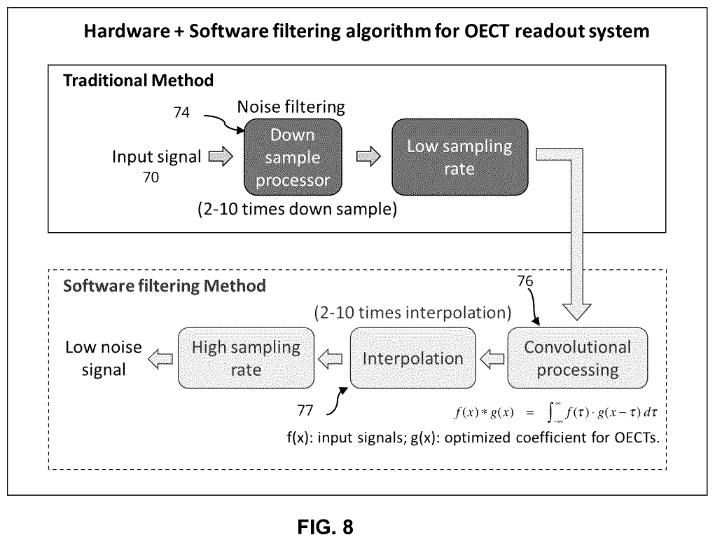

To solve challenge 3, the present invention uses a novel algorithm to increase the sampling rate. Conventional approaches to characterization of OECTs have a very low sampling rate due to the lack of an efficient filtering algorithm. The filtering can be performed solely with hardware, but this method causes a low sampling rate. To solve this challenge, a novel filtering algorithm is used where software filtering is employed to complement hardware filtering (detailed in FIG. 8). The weight of software filtering and hardware filtering is specifically controlled to achieve a high sampling rate. With this method, a high sampling rate of 200 k/s is achieved. It has been verified that this new algorithm can enable a high sampling rate capable of OECT characterizations without sacrificing signal quality.

To solve challenge 4 a pin multiplexing methodology is used to achieve EC-OECT dual mode measurement without too much of an increase in circuit area and power consumption. The PERfECT system can also be converted to a miniaturized electrochemical (EC) station because of an integrated potentiostat amplifier (PA) 12 analytical unit in the voltage control 10, which can help to set the reference electrode. In the NFN circuit part, a programmable switch (SW1) between the feedback loop and amplifier output loop is used for changing the working mode. When used in EC mode, the switch is open and the gate electrode pin and the drain electrode pin of OECTs serve as working and counter electrodes and the EC signal is sampled by the same TIA, ADC and amplifiers. Based on this pin multiplexing, PERfECT can be used in both EC mode and OECT mode but kept at coin size, which is advantageous for data verification and device calibration.

In addition to the above inventive concepts, the following strategies are used to realize a compact and flexible PERfECT platform: To shrink the size of the whole system, state-of-the-art microelectronic components of the smallest size are used. In addition, multilayer manufacturing approaches are used to stack different functional units together. With this method, the size of the whole system is shrunk to one-third of its previous size without losing any accuracy. To further reduce the bulkiness of the system, flexible manufacturing techniques are used to fabricate the system. In addition, a conventional lithium battery is replaced with a flexible paper-based battery to further reduce its weight and bulkiness. The battery is mechanically flexible, and the total thickness is less than 1 mm. Such a compact, lightweight, and flexible platform for OECT characterizations ensures the ability to use if for wearable applications.

The development of the PERfECT platform starts with the circuit design and optimal combination of tiny electronic components that can apply accurate voltage respectively to control drain voltage (V_{d}), source voltage (Vₛ), and gate voltage (V_{g}). It can measure the corresponding Ids with the highest possible resolution. Besides, to characterize an OECT, it is critically required that both the drain-source voltage (V_{ds}) and the gate-source voltage V_{gs} be swept from −1V to +1 V, and the Ids be measured from nA to mA, with a high resolution (<1 nA). Moreover, the physical size of the whole PERfECT system is to be as small as possible so that it can be used for practical wearable applications. FIG. 2A shows the front and back of the platform in comparison with a coin, and the finger of an adult. Because of its small size, the readout is made available remotely, e.g., on a smart phone as shown in FIG. 2A. FIG. 2C shows the device can be worn on the arm or wrist of a user.

To simultaneously satisfy all the above requirements, four sub-modules are used and integrated into the PERfECT system. As shown in FIG. 2B, they include a first module in the form of the voltage control module 10 that contains two digital-to-analog converters (DAC) 11, 13 and potentiostat amplifier (PA) 12 to control the CE Drain voltage which is connected to the pin multiplexing circuit 15 and printed OECT 26. The output of DAC 11 is connected to the non-inverting input of PA 12. A feedback line 16 is provided from the inverting input to the output of PA 12 and to an input RE from multiplexing circuit 15. The output of the PA is connected to the CE Drain terminal of pin multiplexing circuit 15. The output of second DAC 13 is connected to current monitor 20. Accordingly, the CE Drain voltage can be controlled. High-speed 12-bit DACs are used in order to accurately control the output voltage value, which has a high resolution of <1 mV and short tuning time of <1 ms.

The second module is a high accuracy current monitor module 20 which contains the transimpedance amplifier (TIA) that is formed from operational amplifiers 21A and 21B. The output of DAC 13 of voltage control module 10 is connected to the non-inverting input of the TIA 21A and Id is connected to the inverting input of the TIA and the WE Gate is connected to the inverting input of amplifier 21A. The output of the TIA 21A is connected to an input of a multiplexer (MUX) 31, whose output is connected to the ADC 34. Amplifier 21B has its non-inverting input connected to ground and its inverting input connected to a Source signal from a pin of the multiplexing circuit 15. The output of TIA 21B is applied to a second input of MUX 31. Both TIA 21A and 21B have feedback resistors from their outputs to their non-inverting inputs. The TIA amplifiers provide WE Gate and Source signals to MUX 31 which alternately selects them for transmission to ADC 34. Thus, TIAs 21 are used to control the output voltage and convert the input channel current I_{ds} into a voltage value. In particular, the 16-bit ADC 34 is used in order to realize a high current readout resolution of <1 nA.

The third module is a microcontroller (MCU) 30 that controls the working sequences of the DAC, TIA, and ADC to realize the specific characterization mode and enable an adjustable output voltage range between −1.2 V and +1.2 V and a programmable sampling rate of up to 200 k samples per second (SPS). The output of the ADC 34 is connected to an input of the MCU 30 and the MCU has separate outputs to the inputs of each DAC 11, 13 in the voltage control module 10.

A power supply 32 powers the rest of the circuit, i.e. MCU30, MUX 32 and ADC 34. Further, it provides VDD and ground to the device under test.

The fourth module is a wireless communication module 40 connected to MCU 30, which is used to wirelessly connect with the mobile device of FIG. 2A for data exchange and transmission. The mobile device may be any one of a smart phone, notebook tablet, or other wireless mobile device. Notably, by taking advantage of the ultra-low power consumption, a Bluetooth-Low-Power (BLE) chip DA14585 is harnessed in favor of durable operations. After integrating all these modules, the PERfECT system has dimensions less than 1.5 cm*1.5 cm and a weight less than 0.5 gram, which benefits its uses for wearable applications. Because of the small size, the human perceptible readout is on the smart phone. This signal may also be transmitted through the smart phone to a remote data collection and analysis site.

Sensor calibration is mandatory for reliable and accurate signal recording because the performance degradation of biosensors occurs in practical biosensing applications. [18] However, the majority of OECT-based amplifiers are unable to be self-calibrated because the reference resistor in the circuits makes it impossible to control the V_{ds} precisely. [7, 19, 20] This technical challenge is overcome in the system of the present invention by using the potential output control module 10 to control the V_{d} and Vₛ separately. Therefore, the performance of the OECT can be calibrated at any time, which significantly promotes its uses for practical biosensing applications.

The system of the present invention can also be used as a miniaturized electrochemical (EC) station by substituting the printed OECT 26 for the three electrode EC system 36 in FIG. 2B. The EC device has inputs RE, WE and CE which are substituted for the NC, Vs and Vd inputs to the platform for conventional EC characterizations. The PA unit can help establish the reference electrode, which is an indispensable part of EC characterizations. [21] When used in EC mode, the source electrode and the drain electrode will serve as the working electrode (WE) and the counter electrode (CE) in the EC setup (FIG. 10). Rather than use either the OECT or the EC separately, they can be used in parallel. Therefore, the system allows simultaneous monitoring of bio-signals with both EC and OECT mode, which is advantageous because it allows direct comparison of the results obtained with these two techniques and enables facile calibration of the OECT sensor with the EC unit.

Remote OECT Characterizations can be achieved with the system of the present invention. Typical OECT performance characterizations included i) transfer characterization, ii) output characterization, iii) hysteresis characterization, and iv) transient characterization. [2, 22] See FIG. 1C to 1F.

The transfer curve is one of the most important characterizations of OECTs. [23]. It indicates the doping level evolution of the channel with increased V_{gs}. Besides, it can be used to extract secondary performance indicators of the OECTs, such as the on/off ratio and the Gm value at different V_{gs}. [24] When measuring the transfer curve of a typical PEDOT:PSS OECT, the V_{ds} should be fixed at a specific value. The V_{gs} should be able to be scanned between −1 V and +1 V. At the same time, the maximum V_{gs} should not be larger than ±1.23 V to avoid hydrolysis of the water. [7]

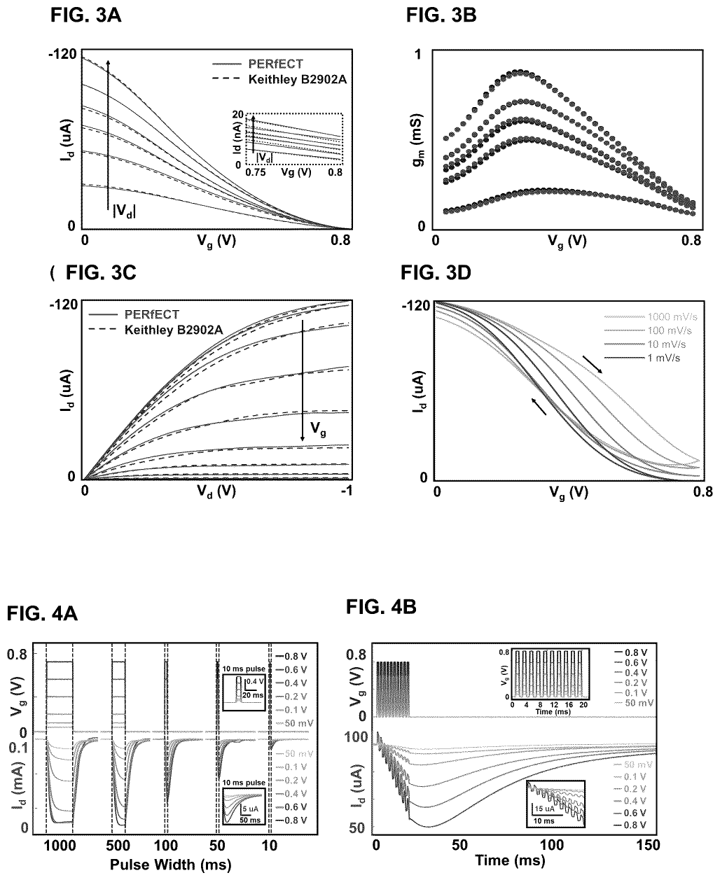

FIG. 3A shows the transfer curve of the same OECT measured by the present invention and a commercial Source Measure Unit (SMU). Identical transfer curves were obtained and demonstrate the high accuracy of the system of the present invention. To further investigate the accuracy of the invention in measuring small current (˜nA), the transfer profiles were also compared at higher Vgs (i.e., highly de-doped channel, lower I_{ds}). It was observed that the system of the present invention can distinguish different I_{ds} values with a high resolution of 1 nA. The accuracy of these values was validated with reference data collected by SMU.

For both the invention and SMU, the Vg is scanned between −0.2 V and 0.8 V, and the Vd is fixed in each curve. The Vg scan step is configured at 5 mV, and each step has a 1.5 S interval. The Vd is changed from −0.1 V to −0.6 V after finishing the Vg scan.

FIG. 3B shows the on/off ratio and transconductance value extracted from the transfer curves recorded by the present invention and SMU. Because of the identical transfer curves recorded with both techniques, a comparable on/off ratio of 100 (Vg1=0 V, Vg2=0.8 V) and an identical transconductance profile are obtained. The OECT had the highest transconductance output at 0.5 mS (Vgs=0.3 V).

The output curve represents the relationship between I_{ds} and channel voltage (V_{ds}) at a constant gate voltage (V_{gs}). [25] It indicates OECT's electrical performances, including the channel's conductivity, working region and the amplification potential. [7, 26, 27] When measuring the output curve of a typical PEDOT:PSS OECT, the V_{gs} should be fixed at a constant value, and V_{ds} can be scanned between −1 V and 0 V. For the output characterization, the V_{d} scan step is 5 mV, and the Vg is changed from 0 V to 0.8 V in steps of 100 mV.

As shown in FIG. 3C, the output curves of the same OECT were measured by both the present invention and SMU. Because of the high resolution in both voltage control and current detection, the output curves measured by the system of the present invention showed a great match with that obtained by the SMU. For example, the I_{ds} difference between the invention and SMU is less than ±1%, regardless of the V_{gs}.

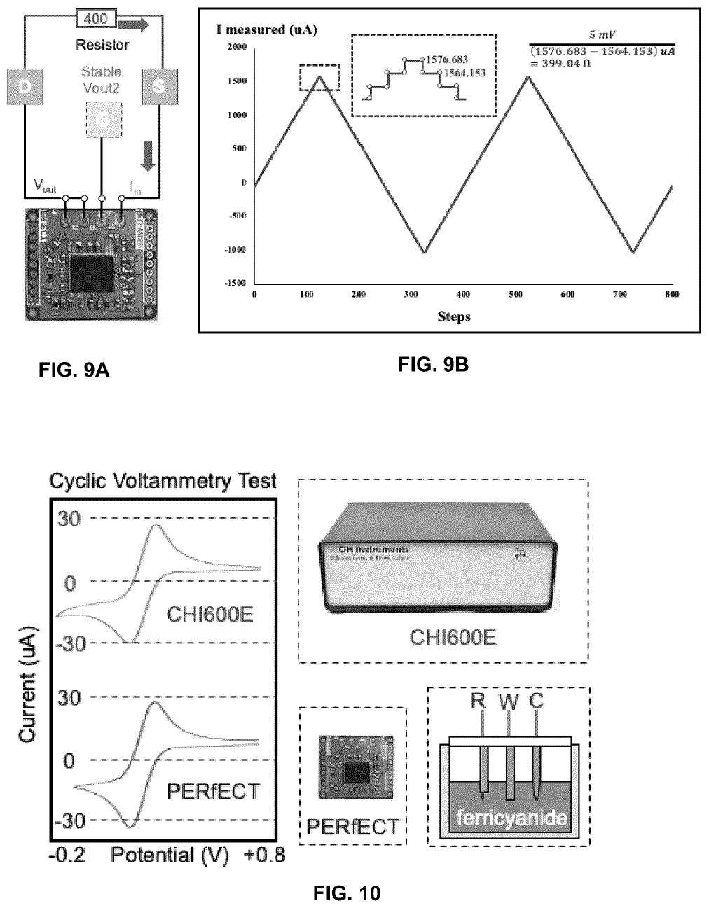

To further investigate the accuracy of the present invention in measuring I_{ds}, i.e., the resistance of the channel, the channel was replaced with a commercial resistor whose resistance is fixed (400 ohms) as shown in FIG. 9A. In this two-terminal device (no V_{gs}), the slope of the curve indicates the resistance value of the resistor. It was observed that both the present invention and SMU obtained the same slope value (sampling rate 200 KSPS), demonstrating the accuracy and high resolution of the invention in controlling V_{ds} and monitoring I_{ds}.

Hysteresis curves are essential in estimating the dynamics behaviors of an OECT, including the ion diffusion speed, the doping kinetics, the synaptic potential, and the device response speed. [28] To measure the hysteresis, both the scanning direction of V_{gs} and the scanning rate should be controllable, which is well covered by the capability of the present invention. In particular, the system of the invention can sweep V_{gs} between −1 and 1 V, with a scanning rate controllable between 1 mV/S and 1 V/S (with a minimum step of 5 mV). FIG. 3D shows the hysteresis curves of OECTs measured with the present invention, where V_{gs} was con-trolled between a typical range (−0.2 V and 0.8 V) with both a positive and negative scan. Hysteresis curves with different scan rates of 1, 10, 100, and 1000 mV/s were presented. The curves of FIG. 3D were generated with V_{ds}=−0.6 V and Vg changed from 0 V to 0.8 V in steps of 5 mV. A larger hysteresis loop was observed at a higher scanning rate, which is consistent with results reported previously, attributable to the suppressed doping/dedoping in the channel. [24]

Transient responses of OECT are frequently used to measure the Ids response speed when a V_{gs} pulse is applied or removed. [29] Therefore, it is a powerful way to benchmark the response speed and frequency response of an OECT. [30] In particular, transient response is the critical parameter in evaluating an OECT's synaptic behavior, which is currently a rising research topic to promote the use of OECT for brain-inspired neuromorphic computing applications. [15-17, 31] However, despite the importance of transient behavior of OECTs, its characterization is more challenging simply because it requires precise control of V_{gs} on both the pulse width and the pulse amplitude. In addition, the duty time, the period, the rest time, and the delay time should be simultaneously controllable to realize a full-spectrum measurement of the transient behavior. [16]

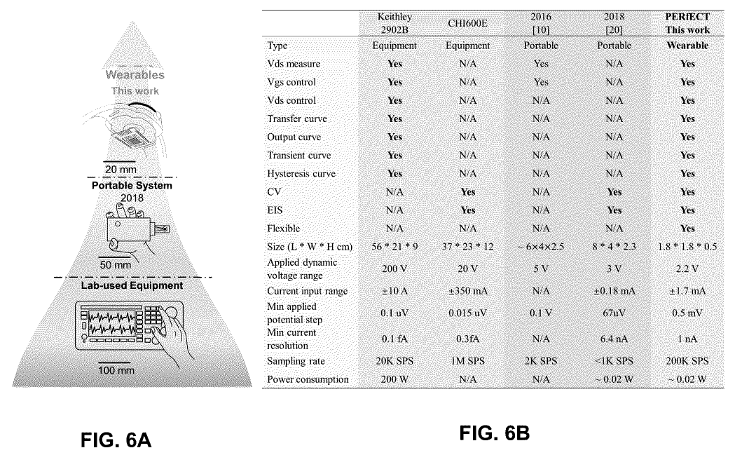

To fulfill the above-mentioned critical requirements, a programmable pulse width function and an arbitrary pulse waveform generation function were incorporated into the system of the present invention without increasing its total dimensions. The updated system allows for the generation of a high-profile pulse (pulse-width down to 1 ms, pulse amplitude controllable between 0.6 mV and 1.5 V). To facilitate the operation, a user-friendly interface was designed that allows the customization of the pulse profiles. As shown in FIG. 4, the system can output precise pulses where the pulse width (FIG. 4A) and pulse amplitude (FIG. 6B) can be accurately controlled. The pulse width can be changed from 10 ms to 1000 ms, and the amplitude can be changed between 50 mV and 800 mV. The I_{ds} responded rapidly to the change of the pulses and expected profiles were recorded.

To validate the potential of using the present invention for the characterization of the transient behavior of OECTs, OECT's synaptic behavior was studied by recoding the I_{ds} change upon the application of defined pulse trains. [16] See FIG. 4B. The I_{ds} shows a distinct decrease with increasing pulse numbers. The increase in pulse amplitude leads to a steeper I_{ds} decrease. These exciting results demonstrate that a system according to the present invention is fully capable of the characterization of the neuromorphic behavior of OECTs, which is relevant for remote and wearable edge-computing applications.

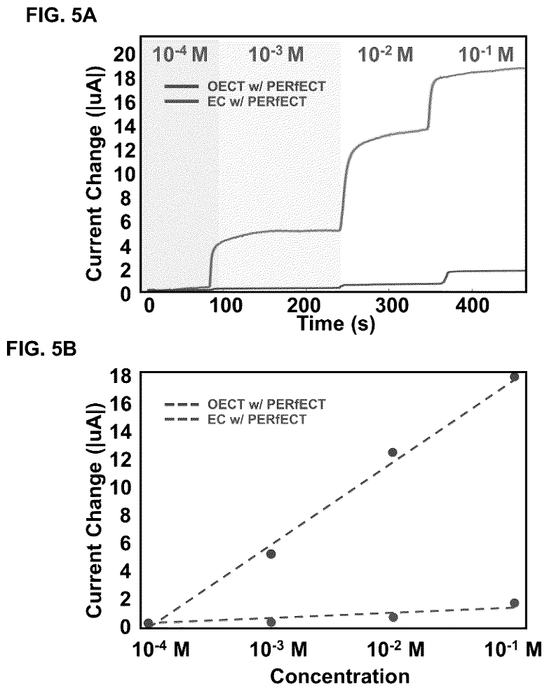

To demonstrate the capability of the present invention for practical applications, a fully integrated wearable bio-sensor was prototyped where the system of the present invention, an OECT sensor and a reference EC electrode were embedded into a watch. Platinum was used as the gate electrode of the OECT for the detection of hydrogen peroxide H₂O₂ molecules using a conventional amperometric EC method. As shown in FIG. 5A, the invention can output a stable I_{ds} of the OECT sensor, and the amperometric current of an EC electrode whose minimum current change is less than 200 nA. Importantly, the invention also indicated that the OECT sensor showed a much higher sensitivity (slope in FIG. 5B) than the conventional EC electrode, thanks to the signal amplification ability of the OECTs.

FIG. 6 shows parameters for previously reported EC/ECT readout systems where NA means “not available.” There has been prior work on the development of portable EC/ECT detectors for a specific application or technique, but they cannot be categorized as a wearable system. Liu et al recently reported a portable OECT readout system for COVID-19 detection but no clear electrical detail can be found for their work. [11] Thus, it seems that the present invention is the first truly wearable system that can wirelessly operate lab-used equipment comparable characterization of both EC and OECT devices.

In summary, the present invention can precisely characterize the transfer curve, output curve, hysteresis loop, and transient response of an OECT. The total manufacturing cost of the device is less than 100 USD, which is acceptable for both research and commercial applications. The figure of merits of the system is comparable to laboratory based commercial bulky equipment. For example, the I_{ds} readout resolution is as low as 1 nA. The data sampling rate is as high as 200 kSPS. These unique properties make the system capable of recoding subtle signals, which paves the way for the use of OECTs for portable and especially wearable sensing applications.

Moreover, the system can also serve as a miniaturized electrochemical station for portable and wearable EC measurements such as cyclic voltammetry. The scanning rate can be controlled between 1 mV/s and 1000 mV/s. This added functionality allows the simultaneous measurement of both EC sensor and OECT biosensor (as illustrated in FIG. 5). Besides, it permits a facile route to calibrate the OECT biosensors by an EC unit, making the whole system competitive for more complex and high-throughput biosensing applications.

Although the present system was developed for OECT characterizations, it has the full capability to measure other kinds of low-voltage transistors, such as electrolyte-gated field-effect transistors (FETs) and high-k dielectric gated thin-film transistors (both FETs and ECTs). Therefore, this platform allows immediate development of more complicated and integrated low-voltage transistor-based circuits and systems for wearable sensing applications. In particular, it can be used to significantly promote the development of wearable edge-computing devices based on emerging low-voltage synaptic transistors, where sensing, data storage, and computing can be realized in a highly miniaturized unit.

FIG. 7 shows a schematic diagram for a negative feedback network (NFN) that can be used for precise voltage control in the present invention. The use an NFN enables high resolution of the system. The NFN in the system has three main parts: a setting voltage source, an operational amplifier and a feedback resistor. [See FIG. 2B] The setting voltage source is generated by a Digital-to-Analog converter (DAC) and then connected to the non-inverting input of the operational amplifier (PA) to provide the base voltage. The feedback resistor [between RE and CE in FIG. 2B] can convert the output current into the feedback voltage and return the voltage into the opposite phase of the operational amplifier. In FIG. 7, Vₒᵤₜ is the output voltage, Vs is the setting voltage, A is the amplifier gain and R is the feedback factor. The entire circuit finally forms a close loop system, and the close-loop gain (G) of the system is:

$$G = \frac{{V}_{out}}{{V}_{s}} = \frac{1}{\frac{1}{β A} + 1}$$

The noise and unexpected error mainly affect the loop gain, L, which is:

$$L = β A > 0$$

Then the G and the dG/dL is:

$$G = \frac{1}{\frac{1}{L} + 1} ,$$

$$\frac{dG}{dL} = \frac{1}{{( L + 1 )}^{2}} ,$$

$$\frac{dG}{G} = \frac{1}{( L + 1 )} \frac{dL}{L} < \frac{dL}{L}$$

Therefore, the NFN can counteract unexpected changes to stabilize the output voltage and for higher resolution.

Down sampling to the raw signal is the traditional denoising method for OECT readout hardware. Suitable times for down sample operation can reduce the high-frequency noise level efficiently. However, the lost samples also decrease the sample rate and result in distortion of the readout results. In order to achieve a high sampling rate while still reducing the noise, additional software filtering is used to complement hardware filtering, which can achieve a balance between the operational speed and sampling rate.

As shown in FIG. 8, there are three main steps for the new filtering method: i) a down sample processing connected to the high sampling rate (800 K/s). An analog-to-digital converter (ADC) 72 first down samples the input signal 70 to quickly remove the high-frequency noise. This can be achieved with down sample processor 74. The down sample times depend on the specific application since different characterizations require different bandwidths. However, the down sample can be 2-10 times. Next, a convolutional-processing-based filtering program 76, which is embedded into the graphic user interface (GUI) software of system, can inhibit both low and high-frequency noise. An interpolation processing module 77, which is a part of the system GUI, can recover the initial sampling rate of the input signal. The result is a high sampling rate signal, but with low noise that can be used for subsequent wireless data transmission.

As noted above, to investigate the accuracy of the invention in measuring Ids, i.e., the resistance of the channel, it is replaced with a commercial resistor whose resistance is fixed (400 ohms). See FIG. 9A. In this two-terminal device (no Vgs), the slope of the curve (FIG. 9B) indicates the resistance value of the resistor. It can be seen that both the present invention and SMU obtained the same slope value (sampling rate 200 KSPS), demonstrating the accuracy and high resolution of the invention in V_{ds} controlling and I_{ds} monitoring.

FIG. 10 shows cyclic voltammetry test results for both the prior art CH1600E SMU and the present invention for electrochemistry. When removing the working mode resistor on the circuit board of the present invention, the system will transfer from OECT mode into electrochemistry (EC) mode. When used in EC mode, the source electrode and the drain electrode serve as working and counter electrodes in the EC setup. Cyclic Voltammetry (CV) is one of the most common EC tests. FIG. 10 shows the comparable CV test result on ferricyanide solution operated by the system of present invention and the CHI600E EC workstation.

### REFERENCES

The cited references in this application are incorporated herein by reference in their entirety and are as follows:
(1) Kim, J.; Campbell, A. S.; De Avila, B. E.-F.; Wang, J. Nature Biotechnology 2019, 37, 389-406.
(2) Rivnay, J.; Inal, S.; Salleo, A.; Owens, R. M.; Berggren, M.; Malliaras, G. G. Nature Reviews Materials 2018, 3, 17086.
(3) Zhang, S.; Hubis, E.; Girard, C.; Kumar, P.; Defranco, J.; Cicoira, F. Journal of Materials Chemistry C 2016, 4, 1382-1385.
(4) Zhang, L.; Wang, G.; Wu, D.; Xiong, C.; Zheng, L.; Ding, Y.; Lu, H.; Zhang, G.; Qiu, L. Biosens Bioelectron 2018, 100, 235-241.
(5) Khodagholy, D.; Rivnay, J.; Sessolo, M.; Gurfinkel, M.; Leleux, P.; Jimison, L. H.; Stavrinidou, E.; Herve, T.; Sanaur, S.; Owens, R. M.; Malliaras, G. G. Nature Communications 2013, 4.
(6) Liu, H.; Zhang, S.; Li, Z.; Lu, T. J.; Lin, H.; Zhu, Y.; Ahadian, S.; Emaminejad, S.; Dokmeci, M. R.; Xu, F.; Khademhosseini, A. Matter 2021.
(7) Braendlein, M.; Lonjaret, T.; Leleux, P.; Badier, J.-M.; Malliaras, G. G. Advanced Science 2017, 4, 1600247.
(8) Bai, L.; Elósegui, C. G.; Li, W.; Yu, P.; Fei, J.; Mao, L. Frontiers in Chemistry 2019, 7.
(9) Tyrrell, J. E.; Boutelle, M. G.; Campbell, A. J. Advanced Functional Materials 2021, 31, 2007086.
(10) Ji, X.; Chan, P. K. L. In Biosensors and Biodetection; Springer New York, 2017, pp 205-216.
(11) Liu, H.; Yang, A.; Song, J.; Wang, N.; Lam, P.; Li, Y.; Law, H. K.-W.; Yan, F. Science Advances 2021, 7.
(12) Khodagholy, D.; Doublet, T.; Quilichini, P.; Gurfinkel, M.; Leleux, P.; Ghestem, A.; Ismailova, E.; Hervé, T.; Sanaur, S.; Bernard, C.; Malliaras, G. G. Nature Communications 2013, 4, 1575.
(13) Keene, S. T.; Fogarty, D.; Cooke, R.; Casadevall, C. D.; Salleo, A.; Parlak, O. Advanced Healthcare Materials 2019, 8, 1901321.
(14) Rivnay, J.; Leleux, P.; Ferro, M.; Sessolo, M.; Williamson, A.; Koutsouras, D. A.; Khodagholy, D.; Ramuz, M.; Strakosas, X.; Owens, R. M.; Benar, C.; Badier, J.-M.; Bernard, C.; Malliaras, G. G. Science Advances 2015, 1, e1400251.
(15) Van De Burgt, Y.; Lubberman, E.; Fuller, E. J.; Keene, S. T.; Faria, G. C.; Agarwal, S.; Marinella, M. J.; Alec Talin, A.; Salleo, A. Nature Materials 2017, 16, 414-418.
(16) Ji, X.; Paulsen, B. D.; Chik, G. K. K.; Wu, R.; Yin, Y.; Chan, P. K. L.; Rivnay, J. Nature Communications 2021, 12.
(17) Fuller, E. J.; Keene, S. T.; Melianas, A.; Wang, Z.; Agarwal, S.; Li, Y.; Tuchman, Y.; James, C. D.; Marinella, M. J.; Yang, J. J.; Salleo, A.; Talin, A. A. Science 2019, 364, 570-574.
(18) Bandodkar, A. J.; Jeerapan, I.; Wang, J. ACS Sensors 2016, 1, 464-482.
(19) Zhang, S.; Ling, H.; Chen, Y.; Cui, Q.; Ni, J.; Wang, X.; Hartel, M. C.; Meng, X.; Lee, K.; Lee, J.; Sun, W.; Lin, H.; Emaminejad, S.; Ahadian, S.; Ashammakhi, N.; Dokmeci, M. R.; Khademhosseini, A. Advanced Functional Materials 2020, 30, 1906016.
(20) Ji, X.; Lau, H. Y.; Ren, X.; Peng, B.; Zhai, P.; Feng, S.-P.; Chan, P. K. L. Advanced Materials Technologies 2016, 1, 1600042.
(21) Ainla, A.; Mousavi, M. P. S.; Tsaloglou, M.-N.; Redston, J.; Bell, J. G.; Fernindez-Abedul, M. T.; Whitesides, G. M. Analytical Chemistry 2018, 90, 6240-6246.
(22) Friedlein, J. T.; McLeod, R. R.; Rivnay, J. Organic Electronics 2018, 63, 398-414.
(23) Tan, S. T. M.; Keene, S.; Giovannitti, A.; Melianas, A.; Moser, M.; Mcculloch, I.; Salleo, A. Journal of Materials Chemistry C 2021.
(24) Kumar, P.; Yi, Z.; Zhang, S.; Sekar, A.; Soavi, F.; Cicoira, F. Applied Physics Letters 2015, 107, 053303.
(25) Tybrandt, K.; Zozoulenko, I. V.; Berggren, M. Science Advances 2017, 3, eaao3659.
(26) Venkatraman, V.; Friedlein, J. T.; Giovannitti, A.; Maria, I. P.; McCulloch, I.; McLeod, R. R.; Rivnay, J. Advanced Science 2018, 5, 1800453.
(27) Tyrrell, J. E.; Petkos, K.; Drakakis, E. M.; Boutelle, M. G.; Campbell, A. J. Advanced Functional Materials 2021, 2103385.
(28) Giovannitti, A.; Sbircea, D.-T.; Inal, S.; Nielsen, C. B.; Bandiello, E.; Hanifi, D. A.; Sessolo, M.; Malliaras, G. G.; Mcculloch, I.; Rivnay, J. Proceedings of the National Academy of Sciences 2016, 113, 12017-12022.
(29) Bernards, D. A.; Malliaras, G. G. Advanced Functional Materials 2007, 17, 3538-3544.
(30) Rivnay, J.; Ramuz, M.; Leleux, P.; Hama, A.; Huerta, M.; Owens, R. M. Applied Physics Letters 2015, 106, 043301.
(31) Melianas, A.; Quill, T. J.; Lecroy, G.; Tuchman, Y.; Loo, H. V.; Keene, S. T.; Giovannitti, A.; Lee, H. R.; Maria, I. P.; McCulloch, I.; Salleo, A. Science Advances 2020, 6, eabb2958.

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. An electronic reader for characterizing electrochemical devices comprising:
a voltage output control module that contains at least two digital-to-analog converters (DAC) and a potentiostat amplifier (PA) to control the inputs for the device under test, one DAC output is connected to the non-inverting input of the PA, a feedback line is connected to the inverting input of the PA, the output of the PA is connected to Vs (CE Drain) and a reference electrode (RE);
a high accuracy current monitor module which contains a transimpedance amplifier (TIA) and an analog-to-digital converter (ADC), wherein the TIA is used to control the output voltage and convert the input channel current I_{ds} into a voltage value, the second DAC of the voltage output control module is connected to the non-inverting input of the TIA, the output of the TIA is connected to an input of the ADC; and
a microcontroller (MCU) that controls the working sequences of the DAC, TIA, and ADC to realize the specific characterization mode and enable an adjustable output voltage range, wherein said MCU further controls a programmable sampling rate (SPS), and the MCU has separate outputs to the inputs of each DAC in the output control module.

2. The electronic reader according to claim 1, further including a wireless communication module which is used to connect the MCU with a remote device for data exchange and transmission.

3. The electronic reader according to claim 2, wherein the remote device is a wireless mobile device and the wireless communication module is a Bluetooth-Low-Power (BLE) chip that communicates with a Bluetooth circuit in the mobile device.

4. The electronic reader according to claim 1 wherein the DACs are high-speed 12-bit DACs which allow accurate control of the output voltage value with a high resolution down to <1 mV and a short tuning time down to <1 ms.

5. The electronic reader according to claim 1 wherein the ADC in the current monitor is a 16-bit ADC in order to realize a high current readout resolution of down to <1 nA.

6. The electronic reader according to claim 2 located on a chip with dimensions less than 1.5 cm*1.5 cm and a weight of less than 0.5 gram, which benefits its uses for wearable applications.

7. The electronic reader according to claim 1 wherein the readout resolution is improved and the voltage of the reference electrode is precisely controlled by introducing a negative feedback network (NFN) about the PA.

8. The electronic reader according to claim 1 wherein the MCU is programmed with an efficient software algorithm to increase the sampling rate while filtering in order to reducing noise, comprising the steps of:
down sampling the input signal by 2 to 10 times to remove high-frequency noise and achieve a low sampling rate signal;
convolutional-processing the low sampling rate signal to produce a high sampling rate signal with low noise; and
interpolation processing the convolutional processed signal to recover the initial sampling rate of the input signal without low or high frequency noise.

9. The electronic reader according to claim 8 wherein the down sampling is achieved with an analog to digital converter and a down sample processor, the convolutional processing is a filtering process embedded in a graphical user interface of the software to inhibit both low and high-frequency noise.

10. The electronic reader according to claim 8 wherein the filtering of the software algorithm is employed as a complement to hardware filtering, and the weight of software filtering and hardware filtering is specifically controlled to achieve a high sampling rate.

11. The electronic reader according to claim 1 further including a flexible paper-based battery to power the reader while providing it with reduced weight and bulkiness.

12. The electronic reader according to claim 1 wherein the transimpedance amplifier comprises a multiplexer (MUX) and first and second operational amplifiers,
wherein the first operational amplifier has is non-inverting input connected to the output of the second DAC, its output applied to one input of the MUX and its inverting input connected to a WE Gate signal;
wherein the second operational amplifier has its non-inverting input connected to ground, its output applied to another input of the MUX and its inverting input connected to a source signal, and
the MUX output alternately applies the outputs of the transimpedance amplifiers to the input of the ADC under the control of the MCU.

13. The electronic reader according to claim 1, wherein the devices are organic electro-chemical transistors (OECT) and the potentiostat amplifier (PA) controls the inputs V_{d}, Vₛ, and V_{g} for the OECT.

14. The electronic reader according to claim 13 for an organic electro-chemical transistor (OECT) is further used as a miniaturized electrochemical (EC) station by substituting a three electrode EC system for the OECT where inputs RE, WE and CE of the EC system replace the Vs, Vg and Vd inputs,
whereby EC and OECT characterization can be performed in the same circuit, and
whereby the PA unit helps establish the reference electrode, the source electrode and the drain electrode serve as the working electrode (WE) and the counter electrode (CE).

15. The electronic reader of claim 14 wherein use the characterization of the OECT or the EC is performed separately under the control of a programmable switch.

16. The electronic reader of claim 14 wherein the characterization of the OECT and the EC are performed in parallel simultaneously by using pin multiplexing, whereby direct comparison of the results of these two techniques can be compared and facile calibration of the OECT sensor with the EC unit is enabled.

17. The electronic reader of claim 15, wherein the scanning rate can be controlled between 1 mV/s and 1000 mV/s.

18. The electronic reader according to claim 14 located on a chip with dimensions less than 1.5 cm*1.5 cm, which benefits its uses for wearable applications and allows for dual mode measurements on a single chip.

19. A method for fabricating an organic electrochemical transistor (OECT) on plastic substrates, comprising the steps of:
pre-pattering source, drain, and gate electrodes on plastic substrates in a planar substrate structure;
mixing a PEDOT:PSS suspension with surfactant dodecyl benzene sulfonic acid (DBSA) (0.5 v/v. %) and crosslinker 3-glycidoxypropyltrimethoxysilane (GOPS) (1 v/v. %) to improve the wettability and adhesion on the substrate;
spin-coating and patterning the PEDOT:PSS suspension mixture between the source and drain electrodes;
using Kapton tape as a show mask for the patterning of the channel;
baking the structure on a hotplate for about 1 hour to anneal the PEDOT:PSS channel into a film;
soaking the structure in deionized water to remove saline contaminants from the PEDOT:PSS channel film;
applying a solid-state ion gel as an electrolyte bridging the gate electrode and the PEDOT:PSS channel.

20. The method for fabricating an organic electrochemical transistor of claim 19 wherein the ion gen is formed by a process comprising the steps of:
performing a one-step polymerization comprising the steps of:
mixing a zwitterionic monomer 3-dimethyl (methacryloyloxyethyl) ammonium propane sulfonate (DMAPS) with ionic liquid (1-ethyl-3-methylimidazolium ethyl sulfate) deionized water with a weight ratio of 1:1:4.67, and
initializing the mixture with ammonium persulfate (APS) at 70° C. for 6 hours, and
curing the mixture in a 50° C. oven to remove excess water and to obtain the ion gel.

21. The method for fabricating an organic electrochemical transistor of claim 19 wherein the plastic substrates are 3M Tegaderm Roll.

22. A method to improve the control and reading resolution of the electronic reader of claim 1 by introducing a negative feedback network (NFN) about the PA.

23. Microneedles for biosensing having the electronic reader of claim 1 integrated therein.

24. A smartwatch for wearable sensing having the electronic reader of claim 1 integrated therein.
